(19) United States
(12) Patent Application Publication
(10) Pub. No.: US 2018/0296364 A1
(43) Pub. Date: Oct. 18, 2018

(54) METHOD AND APPARATUS FOR ASSEMBLING MODULAR PROSTHETIC COMPONENTS

(71) Applicants: Evan Harris, San Antonio, TX (US); Adam I. HARRIS, San Antonio, TX (US)

(72) Inventors: Evan Harris, San Antonio, TX (US); Adam I. HARRIS, San Antonio, TX (US)

(21) Appl. No.: 15/486,989

(22) Filed: Apr. 13, 2017

(51) Int. Cl. A61F 2/46

(52) U.S. Cl. CPC A61F 2/4607; A61F 2002/30604

(57) ABSTRACT

An assembly tool for affixing a modular femoral head prosthesis to a femoral stem prosthesis during total hip arthroplasty may be provided. The assembly tool may be utilized in conjunction with prosthetic femoral components having a Morse taper arrangement. A predetermined amount of force may be calculated to properly set the prosthetic components. The assembly tool may include hemicylindrical bearings, a clamp, and an impaction cap having extension members connected thereto. The hemicylindrical bearings may be mounted on the neck portion of the femoral stem prosthesis and removably secured thereto via a vice-like clamp. Extension members may attach to the hemicylindrical bearings and extend around the femoral head to an impaction cap. The impaction cap may be mounted on the femoral head and provide the linear biasing force necessary to secure the femoral head to the femoral stem.

### BACKGROUND

The hip joint is one of the major weight-bearing joints in our body, assuming distributional stresses from both static (e.g., standing) and dynamic (e.g., walking) activities. Standing on two legs, for example, loads the hip joint with a force equivalent to 30% body weight, while forces exerted during walking can range from approximately 2-4 times the body weight. Normally, the hip functions as a relatively frictionless “ball-and-socket” joint enclosed by a ligamentous capsule. The rounded head of the femur forms the ball, which rotates within a cup-shaped socket, or the acetabulum, located in the pelvis. Articular cartilage completely covers the bone surface inside the joint, providing a smooth and lubricated surface for articulation. The cartilage also acts as a flexible shock absorber to prevent the impact of contiguous bone.

Despite its ability to withstand repeated loading forces, the hip joint can deteriorate over time due to various degenerative diseases, injuries, and aging. Osteoarthritis, for example, is the most common joint disorder affecting over 20 million individuals in the United States. The disease leads to the progressive deterioration of articular cartilage between the femoral head and the acetabulum. Eventually, the smooth cartilage that normally cushions adjacent bony surfaces wears down, causing severe pain, stiffness, instability, and restriction of motion in the hip. Other common causes of chronic hip pain and disability include inflammatory arthritis (e.g., rheumatoid or psoriatic arthritis), hip disorders of infancy and childhood, osteonecrosis (avascular necrosis), and trauma.

When the natural hip joint becomes sufficiently damaged or diseased, the defective bone and cartilage can be removed and replaced with artificial material. Total hip arthroplasty is the surgical replacement of the hip joint with a prosthetic implant. Introduced by Sir John Charnley in the early 1960s, the treatment aims to restore the functionality of leg movement and alleviate hip pain that cannot be remedied through non-operative procedures. Typically, reconstruction of the hip joint is accomplished with two prosthetic hip components, the femoral component and acetabular component, which replace the natural femoral head and acetabulum, respectively.

Total hip arthroplasty usually involves the surgical excision of the head and proximal neck of the femur, acetabular cartilage and subchondral bone. A femoral prosthesis, having an articulating femoral head attached to an elongated stem, is implanted within the femoral intramedullary canal. At an enlarged acetabular space, an acetabular prosthesis forming a hemispherical cup with low-friction articulating surface is secured to the native pelvic bone. These implants, necessarily constructed from biocompatible material, are coupled together via articulating bearing surfaces to define the final prosthesis. The coupling should maintain the prosthetic components in a position that closely replicates the natural hip joint, thereby simulating natural joint kinematics and facilitating near natural movement.

To ensure successful restoration of hip functionality, it is crucial that the hip implant properly align with the surrounding bone. Various modular prosthetic components have been developed to ensure a customized fit for all variations in patient anatomy. Modular components allow surgeons greater intraoperative flexibility, which helps to minimize incision length and surgical dissection. There is also longstanding and well-established technology for the design of the interference fit between prosthetic components. The Morse taper consists of two assembly segments, a male taper (trunnion) and a female taper (bore), which mate together to securely join modular components. A lockable attachment between the femoral stem (male taper) and femoral head (female taper) is accomplished by the friction forces exerted on trunnion and bore surfaces.

Unfortunately, traditional techniques to implant a modular hip prosthesis have well-recognized shortcomings. In particular, the current method of affixing a modular head to a femoral stem during total hip arthroplasty is the use of a mallet and a striking device that transmits the impaction force. Impaction of the head upon the stem causes some deformity of one or both tapers (depending on the material) locking the components to each other. The force of impact is quite variable and surgeon dependent. Short term survivorship of the implant depends on avoiding excessive force which could dislodge the femoral stem or fracture the proximal femur. Long term survivorship depends on adequate impaction so as to minimize the possibility of micromotion at the junction.

Under optimal circumstances, the interface should be able to withstand torsional forces multiplied over several million expected cycles without breakdown. However, should breakdown occur and micromotion ensue, corrosion of a metal-on-metal interface can cause significant local and occasionally systemic issues. If the prosthetic head is made from ceramic, micromotion increases the risk of fracture.

Furthermore, considerable force is required to create an adequate interface. The force must also be in line with the longitudinal axis of the femoral neck, which is offset from the axis of the prosthesis body by an average of 45-degrees. Surgeons are often reticent to strike with adequate force as many of the candidates for total hip arthroplasty are elderly. A force too excessive may cause a premature femoral fracture.

Current methodologies are aimed at determining the adequate striking force and then training surgeons to impact with adequate force via lab simulators. However, these methodologies cannot assess impaction forces during an actual surgery.

### SUMMARY

Exemplary embodiments described herein generally relate to implantable prosthetic devices, and, more specifically, to a method and apparatus for assembling modular orthopedic prosthetic components. An assembly tool for affixing a first prosthetic component to a second prosthetic component during joint arthroplasty may be provided. The assembly tool may be adapted for compressing a self-locking taper junction between first and second prosthetic components. Examples of such prosthetic components may include artificial joints for the knee, elbow, hip, shoulder, ankle, and wrist.

According to an exemplary embodiment, an assembly tool for affixing a modular femoral head prosthesis to a femoral stem prosthesis during total hip arthroplasty may be provided. The assembly tool may be utilized in conjunction with prosthetic femoral components having a Morse taper arrangement. In particular, a femoral stem prosthesis having a tapered male connection may interconnect with a femoral head prosthesis having a female taper connection. The femoral stem prosthesis may include a male Morse taper, a neck portion, and an elongated body portion adapted for insertion into a femoral intramedullary canal. The femoral head prosthesis may include a spherical body having a complementary female Morse taper formed in a distally facing surface thereof. The assembly tool may be configured to secure the femoral head prosthesis to the femoral stem prosthesis by delivering a linear biasing force through a calibrated impaction cap. In this way, the assembly tool may obviate the need for conventional mallets or other striking devices, thereby diminishing the risk of femoral component dislodgement and femoral fracture associated with manual impaction.

The assembly tool may include hemicylindrical bearings, a clamp, and an impaction cap having extension members connected thereto. The hemicylindrical bearings may be mounted on the neck portion of the femoral stem prosthesis and removably secured thereto via a vice-like clamp. The clamp may be angled to hold the hemicylindrical bearings in place without disrupting accessibility to the surgical site or the surgeons' line of sight. Extension members may attach to the hemicylindrical bearings and extend around the femoral head to an impaction cap. The impaction cap may be mounted on the femoral head and provide the linear biasing force necessary to secure the femoral head to the femoral stem. The impaction cap may include a cylindrical housing having a bore with internal threads, and a screw member having complementary external threads disposed therein. A fitting for a torque wrench may be provided on an exterior surface of the impaction cap; engagement of the fitting with a torque wrench may facilitate displacement of the screw member from inside the housing. A predetermined amount of force may be calculated for assembling prosthetic components. The torque wrench may be calibrated to read the external forces exerted thereon, and determine when the force is sufficient to lock the Morse taper arrangement.

### BRIEF DESCRIPTION OF THE DRAWINGS

Advantages of embodiments of the present invention will be apparent from the following detailed description of the exemplary embodiments. The following detailed description should be considered in conjunction with the accompanying figures in which:

FIG. 1 shows a side perspective view of an exemplary embodiment of a femoral stem prosthesis for an artificial hip joint;

FIG. 2 shows a side perspective view of an exemplary embodiment of a femoral head prosthesis configured to interface with the femoral stem prosthesis of FIG. 1;

FIG. 3 shows a side perspective view of an exemplary embodiment of a modular femoral prosthetic implant;

FIG. 4 shows a side perspective view of an exemplary embodiment of an assembly tool for affixing a modular femoral head prosthesis to a femoral stem prosthesis; and

FIG. 5 shows a side perspective view of an exemplary embodiment of a screw member for use with an impaction cap.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

An apparatus for assembling modular orthopedic prosthetic components may be described herein. According to an exemplary embodiment, an assembly tool for affixing a first prosthetic component to a second prosthetic component during joint arthroplasty may be provided. The assembly tool may be adapted for compressing a self-locking taper junction between first and second prosthetic components. Examples of such prosthetic components may include artificial joints for the knee, elbow, hip, shoulder, ankle, and wrist.

In an exemplary embodiment, an assembly tool may be used to affix a modular prosthetic femoral head to a prosthetic femoral stem during total hip arthroplasty. The assembly tool may be utilized in conjunction with prosthetic femoral components having a Morse taper arrangement. In particular, a femoral stem prosthesis having a tapered male connection may interconnect with a femoral head prosthesis having a female taper connection. The assembly tool may be configured to secure the femoral head prosthesis to the femoral stem prosthesis by delivering a controlled linear biasing force through a calibrated impaction cap. In this way, the assembly tool may obviate the need for conventional mallets or other striking devices, thereby diminishing the risk of femoral component dislodgement and femoral fracture associated with manual impaction.

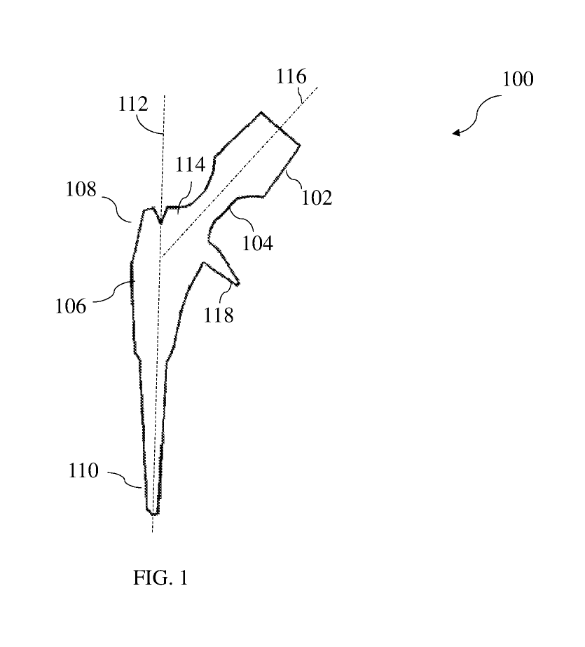

Referring now to the drawings, and more particularly to FIG. 1, an exemplary embodiment of a femoral stem prosthesis 100 for an artificial hip joint may be shown. The femoral stem prosthesis 100 may be configured to be inserted into the intramedullary canal of the femur. Prior to implantation, a surgeon may make an incision to access and dislocate the hip joint, exposing the articulating bone ends. Damaged femoral cartilage and bone may then be extracted from the natural femur, and the intramedullary canal prepared to receive the prosthesis. In particular, the intramedullary bone space may be carved out to create a cavity that matches the shape of the femoral stem prosthesis. The stem prosthesis may then be inserted into the prepared intramedullary canal, and affixed thereto via any known adhesion method as would be understood by a person having ordinary skill in the art. In some exemplary embodiments, for example, the joint prosthesis may be affixed to the femur by the application of a fast-drying bone cement. In other exemplary embodiments, the prosthesis may be specially textured or constructed of porous material that allows bone ingrowth over time.

The femoral stem prosthesis 100 may include a male Morse taper 102, a neck 104, and an elongated stem 106. The elongated stem 106 may convergently taper from a proximal end 108 to a distal end 110 along a first longitudinal axis 112. In some exemplary embodiments, the elongated stem may further include anterior and posterior locking surfaces for impaction of the stem into the femur. The locking surfaces may form indentations or cavities within the surface of the stem, including but not limited to through-slots, deep grooves, tunnels, or pits.

The neck portion 104 of the femoral stem prosthesis 100 may protrude from a juncture 114 at the proximal end 108 of the stem, and extend along a second longitudinal axis 116 oblique to the first 112. In some exemplary embodiments, for example, the second longitudinal axis 116 may be offset from the first longitudinal axis 112 by 45 degrees. The neck 104 may terminate in a conical frustum, or male Morse taper 102, designed to frictionally interlock with a corresponding female Morse taper of the femoral head prosthesis. The width of the male Morse taper 102 may be larger than the width of the adjoining neck region 104. The diameter of the male Morse taper 102 may narrow from about 14 millimeters to about 12 millimeters.

In some exemplary embodiments, the femoral stem prosthesis 100 may further include a flange portion 118 extending along the juncture 114 between the stem 106 and neck 104. The flange 118 may thus give the femoral stem prosthesis 100 an L-shaped appearance, the vertical component representing the elongated stem 106 and the horizontal component representing the flange 118. Alternatively, a number of flanges may extend along the juncture in diametrically opposing directions, giving the femoral stem prosthesis 100 a T-shaped configuration. When the femoral stem prosthesis 100 is inserted into the intramedullary canal, the flange portion 118 may engage a proximally-facing resected surface of the femoral neck. The flange 118 may thus assist in maintaining the position of the prosthesis 100 within the intramedullary canal and distribute physiological forces to the upper portion of the resected femur. The angle, size, and extent of the flange portion 118 may depend on the anatomy of the patient and the morphology of the femoral resection as would be understood by a person having ordinary skill in the art.

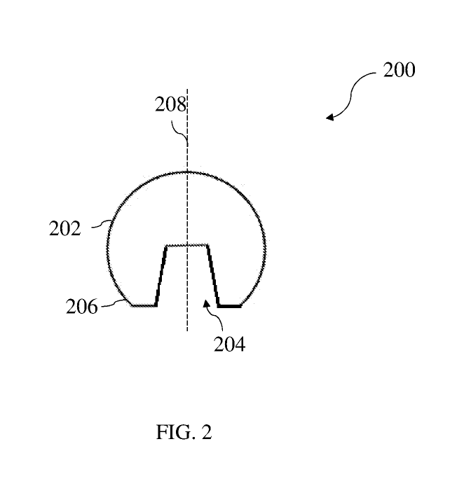

FIG. 2 may depict an exemplary embodiment of a femoral head prosthesis 200 configured to interface with the femoral stem prosthesis of FIG. 1. The femoral head prosthesis 200 may include an approximately spherical body 202 having a complementary female Morse taper 204 formed in a distally facing surface 206 thereof. The spherical body 202 may have an external bearing surface designed to articulate within an acetabulum (not shown) with relatively low friction. The acetabulum surface may be a natural socket, such as in a hip hemiarthroplasty, or may be an artificial acetabular cup as is the case for a total hip arthroplasty. The female Morse taper 204 may extend within the spherical body to form a conically tapered recess. The recess, configured to mate with a male Morse taper, may be concentric with an axis of symmetry 208 for the spherical body 202.

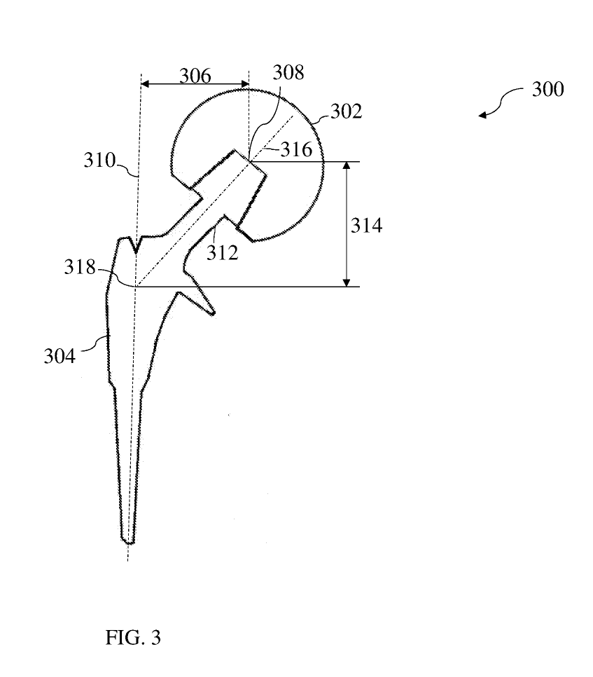

FIG. 3 may illustrate an exemplary embodiment of a modular femoral prosthetic implant 300 with femoral head 302 secured to the femoral stem 304. The spherical body of the femoral head 302 may be spaced apart from the femoral stem prosthesis 304 by a lateral offset distance 306, representing the distance between the center of rotation 308 of the femoral head 302 and the first longitudinal axis 310 of the femoral stem 304. The distance between the femoral head 302 and the femoral stem 304 provided by the femoral neck 312 may be represented as the leg length 314. The leg length 314 may be determined as the vertical distance between the center 308 of the femoral head 302 and the intersection of the first longitudinal axis 310 and second longitudinal axis 316 at focal point 318. The second longitudinal axis 316 may extend through the neck 312 and the center of the femoral head 302. Proper hip functioning may be attained by selecting an appropriate offset value 306 and leg length 314 to match patient anatomy. A longer neck portion 312, for example, will necessarily increase the lateral offset distance 306 and leg length 314 since the center 308 will be positioned farther away from the first longitudinal axis 310. Accordingly, prosthetic components with predetermined lateral offset distance 306 and leg length 314 most closely resembling natural patient anatomy may be used in order to optimize hip mechanics.

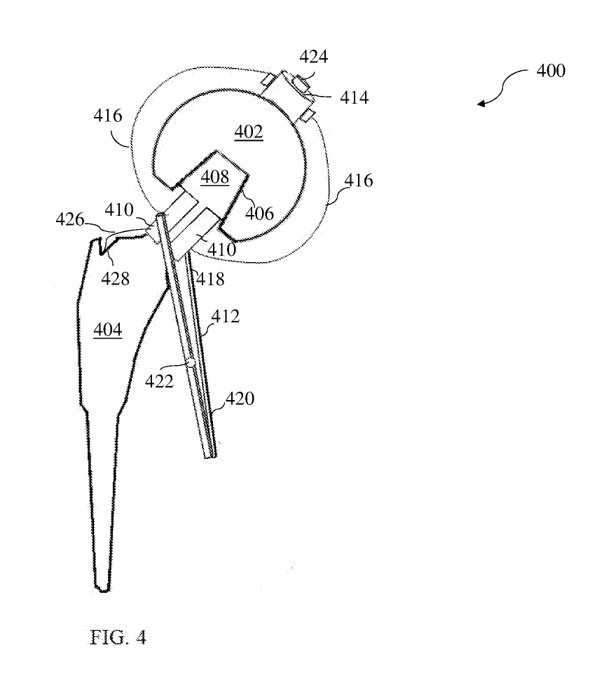

FIG. 4 may illustrate an exemplary embodiment of an apparatus 400 for assembling a multicomponent orthopedic prosthesis. The assembly tool 400 may be configured to secure a modular femoral head prosthesis 402 to a femoral stem prosthesis 404 during hip replacement surgery. The assembly tool 400 may engage the two prosthetic components and maintain them at proper angular orientation to create an adequate interface. The femoral head prosthesis 402 may be placed in communication with the femoral stem prosthesis 404 by engaging the female Morse taper 406 with the male Morse taper 408. The assembly tool 400 may then provide a sustained linear biasing force along a properly oriented longitudinal axis, so as to create localized deformation of the interference fit between mating connections. In some exemplary embodiments, for example, the assembly tool 400 may facilitate a compression force that is collinear with a central axis of the prosthetic femoral neck. The assembly tool 400 may include hemicylindrical bearings 410, a clamp 412, and an impaction cap 414 having extension members 416 connected thereto.

As shown in FIG. 4, two hemicylindrical bearings 410 may extend from the male Morse taper (trunnion) 408 along the prosthetic femoral neck towards the juncture at the proximal end the femoral stem prosthesis 404. The bearings 410 may be sized and shaped so that, when mounted on the femoral stem prosthesis 404, the bearings 410 do not come into contact with the femoral head prosthesis 402 at any point during assembly. The two bearings 410 may engage and substantially surround the diameter of the neck portion, collectively covering less than 360 degrees so that compression may be achieved between them. Each bearing 410 may be constructed from a metal component having an interior and an exterior surface. The interior surface, abutting the neck portion, may be coated with a surgical grade plastic or similar material so as not to scuff, damage, deform, or degrade the prosthesis. The interior shape of each bearing 410 may be molded to have a shape and curvature that corresponds to the shape and curvature of the prosthetic neck portion. In some exemplary embodiments, for example, the neck portion may intersect the male Morse taper at an approximately 90-degree angle, producing a relatively straight transition along the outer edges of the neck portion to the male Morse tapper (as shown in FIG. 3). In other exemplary embodiments (and as shown in FIG. 1), the outer edges of the neck portion may slightly curve outward, forming a Y-shaped transition from the neck portion to male Morse taper. The bearings 410 may be configured to match the shape and arc of this transition. The exterior surface of each bearing 410 may provide a reinforced metallic backing that supports the interior lining against external compressive forces. The exterior surface may also be adapted to secure the extension members thereto.

The bearings 410 may be removably coupled to the femoral stem prosthesis 404 via a clamp 412. The clamp may firmly attach to an exterior surface of the bearings 410 and grip the bearings 410 against the neck while still allowing compression between them. In some exemplary embodiments, the clamp 412 may be a spring clamp having a plier-like configuration with a gripping end 418, a handle end 420 and a hinge pin 422 therebetween. The gripping end 418 may form a clamping mouth with two clamping jaws that can be spring-loaded toward one another by a biasing spring. The hinge pin 422 may act as a fulcrum and serve to pivotally join the two clamping jaws together. The handle end 420 may be ergonomically shaped with a contoured exterior surface. The angle and position of the clamp against the bearings may be easily manipulated to accommodate different surgical approaches so as not to disrupt accessibility to the surgical site or the surgeon's line of sight.

Extension members 416 may serve as connectors between the bearings 410 and the impaction cap 414. Extension members 416 may also operate as an alignment tool to maintain proper orientation between the bearings 410 and the impaction cap 414 so that the compressive force exerted by impaction cap 414 is collinear with the central axis of the prosthetic femoral neck. In some exemplary embodiments, the assembly tool 400 may utilize one extension member 416 with fixed alignment. To account for differences in the size and shape of the prosthetic hip components, the size and dimension of the bearings 410 may be manipulated via alteration to the size and dimensions of interior plastic coating.

In other exemplary embodiments, and as shown in FIG. 4, the assembly tool may utilize two extension members 416. Each extension member 416 may have a first end and a second end. The first end may attach to an exterior surface of a hemicylindrical bearing 410. Alternatively, the first end of each extension member 416 may attach to the clamp 412 retaining the hemicylindrical bearings 410 against the femoral neck. Each extension member 416 may extend outwardly from its first end around the femoral head 402 without contacting its surface. The second end of each extension member 416 may attach or be geared to the impaction cap 414 mounted on the femoral head prosthesis 402. Extension members 416 may coalesce in a position that is substantially aligned with a longitudinal axis of the attached hemicylindrical bearings 410. The length of each extension member 416 may vary according to the dimensions of the operating femoral head 402. Consequently, the impaction cap 414, when connected to the extension members 416, may be coaxial with the longitudinal axis of the femoral neck. The compressive force, when exerted by the impaction cap 414, may be collinear with the central axis of the prosthetic femoral neck. The extension members 416 will thus maintain proper alignment of the impaction cap 414 for assembling the prosthetic components 402, 404 together.

The impaction cap 414 may be mounted to the femoral head prosthesis and provide the linear biasing force necessary to secure the prosthetic head 402 to the femoral stem 404. The impaction cap 414 may include a cylindrical housing having a bore with internal threads, and a screw member having complementary external threads disposed therein. The screw member may be substantially cylindrical in shape, having an elongated body that extends from a proximal end to a distal end. The distal end may form a concave engagement surface for contacting and engaging with the underlying femoral head prosthesis 402. The engagement surface may be made from surgically compatible material that will not scratch, damage, deform or degrade the exterior surface of the femoral head 402 during engagement.

A fitting 424 for a torque wrench may be provided on an exterior surface of the impaction cap 414. The fitting 424 may take on various configurations known to those skilled in the art for accepting conventional torque wrenches. Engagement of the fitting 424 with a torque wrench may facilitate displacement of the screw member from inside the housing. The torque wrench may be any one of several types of known torque wrenches that tighten fasteners to specified torque levels. The torque wrench may provide a visual, audible, or tactile indication of the amount of applied torque. A “click-type” mechanical torque wrench, for example, may include a calibrated clutch mechanism disposed in a handle of the wrench. When the applied force exceeds a predetermined torque value, the clutch mechanism clicks, generating both an audible sound and tactile sensation of sudden torque release. Other torque wrench arrangements may include beam type, deflecting beam type, slipper type, and electronic strain gauge type indicators.

The torque wrench may be calibrated to read the force exerted on the femoral head prosthesis 402 produced by the displacement of the screw member. The amount of force necessary to properly set the Morse taper may depend on the choice of prosthetic material (e.g., hardness, elasticity), and the difference between radii of the taper connections. A sufficient force may thus be calculated based on these parameters and predetermined before operation. The torque wrench may be used to successively tighten the fitting 424 in order to exert an incrementally stronger force until the predetermined force has been reached, locking the Morse taper arrangement.

The assembly tool 400 may further include a support extension 426 that engages other portions of the femoral stem prosthesis. As shown in FIG. 4, a support extension 426 may attach from a hemicylindrical bearing to a pitted grove 428 within a proximally facing surface of the femoral stem prosthesis 404. The support extension 426 may engage the pitted groove with or without a screw. In other exemplary embodiments, the support extension 426 may engage the flange of the femoral stem prosthesis. The assembly tool 400 may include a single support extension, or may utilize a number of support extensions. The support extension 426 may help to retain the position and proper alignment of the prosthetic components during assembly.

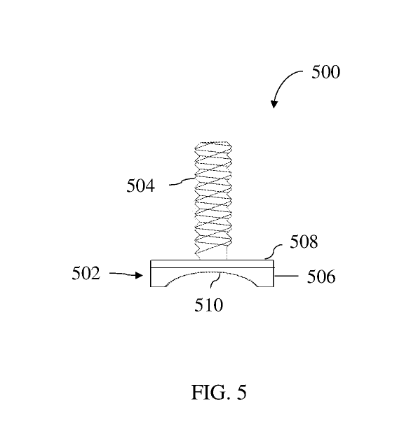

FIG. 5 may show an exemplary embodiment of a screw member 500 configured to slidably engage the cylindrical housing of the impaction cap. The screw member 500 may include a body 502 and a threaded shank 504 defined around a longitudinal axis. The body 502 may have a first side 506 adapted to engage with an underlying femoral head prosthesis and a second side 508 connectable to the thread shank 504. The first side 506 may form a semicircular concave surface 510 designed to mate with and receive a femoral head prosthesis. In some exemplary embodiments, the concave surface 510 may have a radius of curvature that corresponds to that of the spherical bodied head prosthesis. The radius of curvature may range between approximately 11 mm and approximately 22 mm. The surface 510 may be constructed from surgical grade plastic or similar material so as to not scratch, scuff, damage, or deform the femoral head prosthesis during impact. The second side 508 of the body 502 may form a metallic base that supports the threaded shank 504. The threaded shank 504 may extend upwardly from the second side 508 and be co-linear with the radius of curvature of the plastic. The screw member 500 may be interchangeable to accommodate differently sized femoral head prostheses.

In operation, the use of the assembly tool may begin after the femoral stem prosthesis is impacted in the femur, but before final reduction of the new prosthetic head into the acetabulum. A surgeon may place the femoral head prosthesis in communication with the femoral stem prosthesis by engaging the female Morse taper with the male Morse taper. The hemicylindrical bearings may then be placed around the femoral neck. Depending on the type of implant, support extensions may be used to engage the femoral stem at an impaction site. A clamp may then tighten the hemicylindrical bearings against the femoral neck. When first applied, the impaction cap may be backed off from the femoral head. Once the femoral neck is engaged, the impaction cap may be mounted on the femoral head, and connected to the extension members. A torque wrench may then be applied to the impaction cap, forcing the screw member into the femoral head. The torque wrench may have a self-limiting feature that gives way when the applied torque reaches or exceeds the amount of longitudinal compression needed to secure the prosthetic components together.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. An assembly tool for affixing a first prosthetic component to a second prosthetic component, the assembly tool comprising:
a pair of bearings removably coupled to the first prosthetic component;
a clamp for retaining the bearings against the first prosthetic component;
an impaction cap mounted on the second prosthetic component for providing a linear biasing force necessary to secure the second prosthetic component to the first prosthetic component; and
at least one extension member extending from one of the bearings to a position on the impaction cap that substantially aligns with a longitudinal axis of the bearing from which it extends.

2. The assembly tool of claim 1, wherein the impaction cap comprises: a housing having internal threads, and a screw member having complementary external threads disposed therein.

3. The assembly tool of claim 2, wherein the screw member has an elongated body that extends from a proximal end to a distal end, the distal end forming a concave engagement surface.

4. The assembly tool of claim 2, wherein the impaction cap further comprises a torque wrench fitting provided on an exterior surface thereof.

5. The assembly tool of claim 4, wherein engagement of the fitting with a torque wrench facilitates displacement of the screw member from inside the housing.

6. The assembly tool of claim 1, wherein a predetermined amount of force is calculated to properly assemble the first prosthetic component and the second prosthetic component.

7. The assembly tool of claim 5, further comprising a torque wrench calibrated to read a force exerted on the second prosthetic component produced by the displacement of the screw member.

8. The assembly tool of claim 7, wherein the torque wrench provides at least one of: a visual, audible, and tactile indication when an amount of applied torque equals or exceeds a predetermined amount of force necessary to secure the first prosthetic component to the second prosthetic component.

9. The assembly tool of claim 7, wherein the torque wrench is one of: a click-type mechanical torque wrench, a beam type torque wrench, deflecting beam type torque wrench, a slipper type torque wrench, and an electronic strain gauge type torque wrench.

10. The assembly tool of claim 1, wherein the bearings are hemicylindrical.

11. The assembly tool of claim 1, further comprising a support extension that engages a portion of the first prosthetic component.

12. The assembly tool of claim 3, wherein the concave engagement surface has a radius of curvature that corresponds to a radius of curvature of the second prosthetic component.

13. The assembly tool of claim 3, wherein the concave engagement surface is made from a surgical grade plastic.

14. The assembly tool of claim 1, wherein each bearing has an interior surface and an exterior surface, the interior surface being coated with a surgical grade plastic.

15. The assembly tool of claim 1, wherein the first prosthetic component is a femoral stem prosthesis and the second prosthetic component is a femoral head prosthesis.

16. The assembly tool of claim 15, wherein the femoral stem prosthesis comprises a male Morse taper, a neck portion, and an elongated body portion adapted for insertion into a femoral intramedullary canal.

17. The assembly tool of claim 15, wherein the femoral head prosthesis comprises a spherical body having a complementary female Morse taper formed in a distally facing surface thereof.

18. The assembly tool of claim 16, wherein the at least one extension member maintains alignment of the impaction cap so that the linear biasing force is collinear with a central axis of the neck portion of the femoral stem prosthesis.
